(19) United States
(12) Patent Application Publication
(10) Pub. No.: US 2014/0058259 A1
(43) Pub. Date: Feb. 27, 2014

(54) MEASURING DEVICE

(71) Applicant: GE Medical Systems Global Technology Company, LLC, Waukesha, WI (US)

(72) Inventor: Lei Liu, Tokyo (JP)

(73) Assignee: GE MEDICAL SYSTEMS GLOBAL TECHNOLOGY COMPANY, LLC, Waukesha, WI (US)

(21) Appl. No.: 14/011,310

(22) Filed: Aug. 27, 2013

(30) Foreign Application Priority Data
Aug. 27, 2012 (JP) 2012-186146

(51) Int. Cl. A61B 8/08

(52) U.S. Cl. CPC A61B 8/485
USPC 600/438; 600/437

(57) ABSTRACT

A measuring device is provided. The measuring device includes a propagation velocity calculating unit configured to calculate a propagation velocity of a shear wave generated in a living tissue by an ultrasonic pulse transmitted to the living tissue, wherein the propagation velocity calculating unit is configured to calculate propagation velocities of shear waves in a plurality of parts whose distances from the ultrasonic pulse are equal, a comparing unit configured to compare the propagation velocities of the shear waves in the plurality of parts, and a notifying unit configured to perform a notification based on a result of the comparison performed by the comparing unit.

### CROSS REFERENCE TO RELATED APPLICATIONS

This application claims the benefit of Japanese Patent Application No. 2012-186146 filed Aug. 27, 2012, which is hereby incorporated by reference in its entirety.

### BACKGROUND OF THE INVENTION

The present invention relates to a measuring device and an ultrasonic diagnostic apparatus by which elasticity of a living tissue can be known.

A measuring device for measuring an elastic modulus of a living tissue is disclosed in, for example, Japanese Unexamined Patent Application Publication No. 2010-526626. For example, the elastic modulus is calculated as follows. First, an ultrasonic pulse (pushing pulse) for generating a shear wave is transmitted to a living tissue of a subject. Next, propagation velocity of a shear wave generated when the living tissue vibrates by the ultrasonic pulse is calculated.

The propagation velocity of the shear wave and the elastic modulus has a correlation. Specifically, the lower the elastic modulus of a living tissue is (the softer), the higher the propagation velocity of the shear wave is. On the other hand, the higher the elastic modulus of a living tissue is (the harder), the lower the propagation velocity of the shear wave is. Therefore, on the basis of the propagation velocity of the shear wave, the elastic modulus can be calculated.

For example, near a blood vessel or a bone, attenuation of a shear wave is large and the shear wave does not easily propagate. Consequently, there is the case that elasticity of a living tissue is not accurately reflected in the propagation velocity of a shear wave obtained by measurement. In a part which is too far from a body surface, vibration of a living tissue by a pushing pulse is insufficient, and there is a case where propagation velocity of a shear wave in which the elasticity of the living tissue is accurately reflected cannot be obtained. Due to this, there is a case that the elastic modulus calculated on the basis of the propagation velocity of the shear wave is not accurate depending on a place. Therefore, a measuring device or an ultrasonic diagnostic apparatus capable of obtaining more accurate information on the elasticity of a living tissue is in demand.

### BRIEF DESCRIPTION OF THE INVENTION

The embodiments described herein utilize the concept that in places where more accurate elastic moduli can be measured, at least in places whose distances from an ultrasonic pulse causing a shear wave are equal, propagation velocities of shear waves are to be equal to one another.

In a first aspect, a measuring device is provided. The measuring device includes a propagation velocity calculating unit calculating propagation velocity of a shear wave generated in a living tissue by an ultrasonic pulse transmitted to the living tissue, which calculates propagation velocities of the shear waves in a plurality of parts whose distances from the ultrasonic pulse are equal, a comparing unit comparing the propagation velocities of the shear waves in the plurality of parts and a notifying unit performing notification based on a result of comparison performed by the comparing unit.

In a second aspect, a measuring device is provided. The measuring device includes a propagation velocity calculating unit calculating propagation velocity of a shear wave generated in a living tissue by an ultrasonic pulse transmitted to the living tissue, which calculates propagation velocities of the shear waves in a plurality of parts whose distances from the ultrasonic pulse are equal, an elastic modulus calculating unit calculating an elastic modulus of the living tissue corresponding to each of the propagation velocities on the basis of the propagation velocities of the shear waves, a comparing unit comparing the plurality of elastic moduli, and a notifying unit performing notification based on a result of comparison performed by the comparing unit.

In a third aspect, a measuring device is provided. The measuring device includes a propagation velocity calculating unit calculating propagation velocity of a shear wave generated in a living tissue by an ultrasonic pulse transmitted to the living tissue, which calculates propagation velocities of the shear waves in a plurality of parts whose distances from the ultrasonic pulse are equal, an abnormal value specifying unit specifying an abnormal value in accordance with a predetermined reference from the propagation velocities of the plurality of shear waves calculated by the propagation velocity calculating unit, and an average propagation velocity calculating unit calculating an average value of propagation velocities of the shear waves except for the abnormal value.

In a fourth aspect, a measuring device is provided. The measuring device includes an average elastic modulus calculating unit calculating an average elastic modulus on the basis of an average value calculated by the average propagation velocity calculating unit.

In a fifth aspect, a measuring device is provided. The measuring device includes a propagation velocity calculating unit calculating propagation velocity of a shear wave generated in a living tissue by an ultrasonic pulse transmitted to the living tissue, which calculates propagation velocities of the shear waves in a plurality of parts whose distances from the ultrasonic pulse are equal an abnormal value specifying unit specifying an abnormal value in accordance with a predetermined reference from the propagation velocities of the plurality of shear waves calculated by the propagation velocity calculating unit, an elastic modulus calculating unit calculating elastic moduli of the living tissue in the plurality of parts on the basis of the propagation velocities of the shear waves except for the abnormal value and an average elastic modulus calculating unit calculating an average value of the elastic moduli in the plurality of parts.

In a sixth aspect, a measuring device is provided. The measuring device includes a propagation velocity calculating unit calculating propagation velocity of a shear wave generated in a living tissue by an ultrasonic pulse transmitted to the living tissue, which calculates propagation velocities of the shear waves in a plurality of parts whose distances from the ultrasonic pulse are equal, an elastic modulus calculating unit calculating elastic moduli of the living tissue in the plurality of parts on the basis of the propagation velocities of the shear waves, an abnormal value specifying unit specifying an abnormal value in accordance with a predetermined reference from the plurality of elastic moduli, and an average elastic modulus calculating unit calculating an average value of the plurality of elastic moduli except for the abnormal value.

According to the first above-described aspect, propagation velocities of shear waves in a plurality of places whose distances from an ultrasonic pulse are equal are compared and a result of the comparison is notified. Consequently, the operator can know whether a measurement part is proper or not. The operator can therefore perform measurement in a position where more accurate elasticity of a living tissue can be known, and can know more accurate information on the elasticity of a living tissue.

According to the second above-described aspect, elastic moduli of a living tissue obtained in a plurality of places whose distances from an ultrasonic pulse are equal are compared and a result of the comparison is notified. Consequently, the operator can know whether a measurement part is proper or not. The operator can therefore perform measurement in a position where more accurate elasticity of a living tissue can be known, and can know more accurate information on the elasticity of a living tissue.

According to the third above-described aspect, an average value of propagation velocities is calculated on the basis of the propagation velocities of the shear waves except for an abnormal value, so that more accurate information on the elasticity of a living tissue can be obtained.

According to the fourth above-described aspect, an average elastic modulus is calculated on the basis of an average value of propagation velocities of the shear waves except for an abnormal value, so that more accurate information on the elasticity of a living tissue can be obtained.

According to the fifth above-described aspect, the elastic moduli in a living tissue in the plurality of places are calculated on the basis of propagation velocities of the shear waves except for an abnormal value, and an average of the elastic moduli is calculated, so that more accurate information on the elasticity of a living tissue can be obtained.

According to the sixth above-described aspect, an average value of a plurality of elastic moduli other than an abnormal value is calculated, so that more accurate information on the elasticity of a living tissue can be obtained.

### BRIEF DESCRIPTION OF THE DRAWINGS

FIG. 1 is a block diagram illustrating an example of a schematic configuration of a measuring device in a first embodiment.

FIG. 2 is a flowchart illustrating the operation of the first embodiment.

FIG. 3 is a conceptual diagram illustrating a pushing pulse transmitted from an ultrasonic probe and sound rays on which a measurement pulse is transmitted.

FIG. 4 is a diagram illustrating a display unit on which propagation velocity information is displayed.

FIG. 5 is a diagram illustrating a display unit on which propagation velocity information and a message urging re-measurement are displayed.

FIG. 6 is a block diagram illustrating an example of a schematic configuration of a measuring device in a first modification of the first embodiment.

FIG. 7 is a flowchart illustrating the operation of the first modification of the first embodiment.

FIG. 8 is a diagram illustrating a display unit on which average propagation velocity information is displayed in the first modification of the first embodiment.

FIG. 9 is a diagram illustrating a display unit on which average propagation velocity information and a message urging re-measurement is displayed in the first modification of the first embodiment.

FIG. 10 is a block diagram illustrating an example of a schematic configuration of a measuring device in a second modification of the first embodiment.

FIG. 11 is a flowchart illustrating the operation of the second modification of the first embodiment.

FIG. 12 is a diagram illustrating a display unit on which elastic modulus information is displayed in the second modification of the first embodiment.

FIG. 13 is a diagram illustrating a display unit on which elastic modulus information and a message urging re-measurement is displayed in the second modification of the first embodiment.

FIG. 14 is a diagram illustrating a display unit on which propagation velocity information is displayed together with elastic modulus information in the second modification of the first embodiment.

FIG. 15 is a block diagram illustrating an example of a schematic configuration of a measuring device in a third modification of the first embodiment.

FIG. 16 is a flowchart illustrating the operation of the third modification of the first embodiment.

FIG. 17 is a diagram illustrating a display unit on which average elastic modulus information is displayed in the third modification of the first embodiment.

FIG. 18 is a diagram illustrating a display unit on which average elastic modulus information and a message urging re-measurement is displayed in the third modification of the first embodiment.

FIG. 19 is a diagram illustrating a display unit on which average propagation velocity information is displayed together with average elastic modulus information in the third modification of the first embodiment.

FIG. 20 is a block diagram illustrating an example of a schematic configuration of a measuring device in a second embodiment.

FIG. 21 is a flowchart illustrating the operation of the second embodiment.

FIG. 22 is a diagram for explaining positional relations of a pushing pulse and measurement pulses transmitted in the second embodiment.

FIG. 23 is a block diagram illustrating an example of a schematic configuration of a measuring device in a first modification of the second embodiment.

FIG. 24 is a flowchart illustrating the operations of first and second modifications of the second embodiment.

FIG. 25 is a block diagram illustrating an example of a schematic configuration of a measuring device in second and third modifications of the second embodiment.

FIG. 26 is a flowchart illustrating the operation of the third modification of the second embodiment.

FIG. 27 is a block diagram illustrating an example of a schematic configuration of an ultrasonic diagnostic apparatus in a third embodiment.

FIG. 28 is a block diagram illustrating the configuration of a control unit in the ultrasonic diagnostic apparatus in the third embodiment.

FIG. 29 is a block diagram illustrating the configuration of a control unit in an ultrasonic diagnostic apparatus in a first modification of the third embodiment.

FIG. 30 is a block diagram illustrating the configuration of a control unit in an ultrasonic diagnostic apparatus in a second modification of the third embodiment.

FIG. 31 is a block diagram illustrating the configuration of a control unit in an ultrasonic diagnostic apparatus in a third modification of the third embodiment.

FIG. 32 is a block diagram illustrating the configuration of a control unit in an ultrasonic diagnostic apparatus of a fourth embodiment.

FIG. 33 is a block diagram illustrating the configuration of a control unit in an ultrasonic diagnostic apparatus in a first modification of the fourth embodiment.

FIG. 34 is a block diagram illustrating the configuration of a control unit in an ultrasonic diagnostic apparatus in second and third modifications of the fourth embodiment.

### DETAILED DESCRIPTION OF THE INVENTION

Hereinafter, exemplary embodiments will be described with reference to the drawings.

### First Embodiment

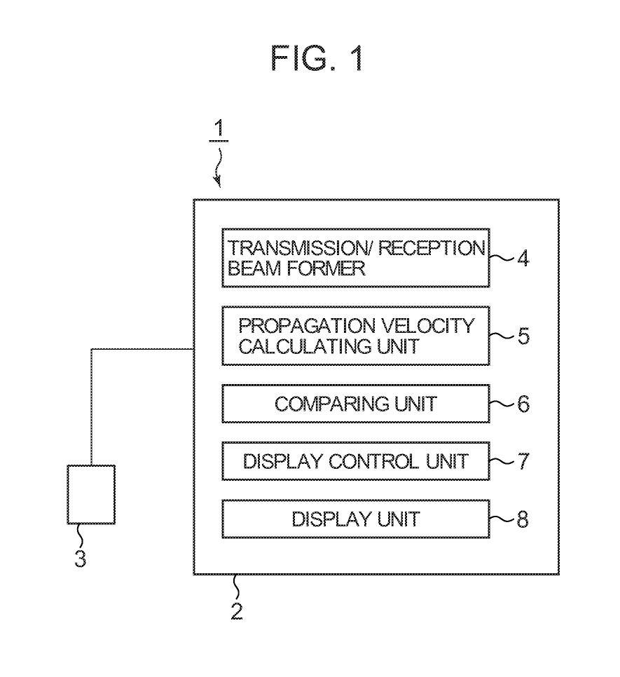

First, a first embodiment will be described with reference to FIGS. 1 to 5. A measuring device 1 illustrated in FIG. 1 has a device body 2 and an ultrasonic probe 3 connected to the device body 2. The device body 2 has a transmission/reception beam former 4, a propagation velocity calculating unit 5, a comparing unit 6, a display control unit 7, and a display unit 8.

The ultrasonic probe 3 transmits an ultrasonic wave to a living tissue in a subject. The ultrasonic wave transmitted includes an ultrasonic wave (pushing pulse) for making the living tissue generate a shear wave and an ultrasonic wave for measuring the propagation velocity of the shear wave. The ultrasonic probe 3 receives an echo signal of the ultrasonic wave transmitted to measure the propagation velocity of the shear wave. The ultrasonic probe 3 is, for example, a 1D array probe having a plurality of ultrasonic transducers arranged in one direction (azimuth direction, X direction).

The transmission/reception beam former 4 transmits the two kinds of ultrasonic waves having a predetermined transmission parameter by making the ultrasonic probe 2 drive. The transmission/reception beam former 4 performs signal processes such as phasing and adding process on the echo signal of the ultrasonic wave transmitted to measure the propagation velocity of the shear wave. As will be described later, the propagation velocity of the shear wave is calculated on the basis of the echo signal subjected to the phasing and adding process.

The propagation velocity calculating unit 5 calculates propagation velocity V of the shear wave generated in the living tissue by the pushing pulse. The propagation velocity calculating unit 5 calculates the propagation velocity V in a plurality of places. The details will be described later. The propagation velocity calculating unit 5 is an example of an embodiment of a propagation velocity calculating unit.

The comparing unit 6 compares the propagation velocities V in the plurality of places calculated by the propagation velocity calculating unit 5. The details will be described later. The comparing unit 6 is an example of an embodiment of a comparing unit.

The display control unit 7 displays propagation velocity information indicative of the propagation velocity V calculated by the propagation velocity calculating unit 5 on the display unit 8. The display unit 8 is, for example, an LCD (Liquid Crystal Display), a CRT (Cathode Ray Tube), or the like. The display unit 8 is an example of an embodiment of a display unit.

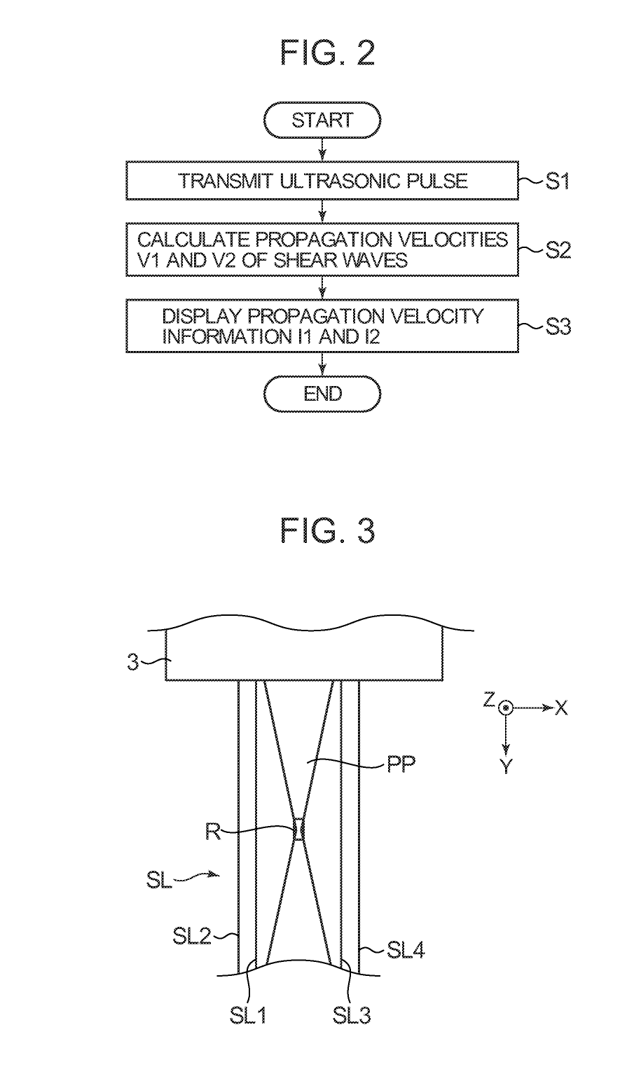

Next, the operation of the embodiment will be described on the basis of the flowchart of FIG. 2. First, in step S1, an ultrasonic pulse is transmitted from the ultrasonic probe 3. The ultrasonic pulse includes a pushing pulse PP and a measurement pulse DP for measuring the propagation velocity of the shear wave.

As illustrated in FIG. 3, the focal point of the pushing pulse PP is in a region R to be measured.

The measurement pulse DP (only sound rays are illustrated in FIG. 2) is transmitted on sound rays SL on both sides of the pushing pulse PP. Specifically, the measurement pulse DP is transmitted on two sound rays SL1 and SL2 on the left side of the pushing pulse PP and two sound rays SL3 and SL4 on the right side.

The distance from the pushing pulse PP to the sound ray SL1 and the distance from the pushing pulse PP to the sound ray SL3 are equal to each other. The distance from the pushing pulse PP to the sound ray SL2 and the distance from the pushing pulse PP to the sound ray SL4 are equal to each other. The distance from the pushing pulse PP can be also said as a distance from the center line of the pushing pulse PP.

The distance between the sound rays SL1 and SL2 and the distance between the sound rays SL3 and SL4 are also equal to each other.

In step S2, the propagation velocity calculating unit 5 calculates propagation velocity V of the shear wave. The propagation velocity calculating unit 5 calculates the propagation velocity V on the basis of the echo signal of the measurement pulse DP received by the ultrasonic probe 3. The propagation velocity calculating unit 5 calculates propagation velocity of a shear wave in one place on the basis of the measurement pulse DP of two sound rays. The propagation velocity calculating unit 5 calculates propagation velocity of shear waves in two places whose distances from the pushing pulse PP are equal to each other.

Specifically, the propagation velocity calculating unit 5 calculates a propagation time difference Δt1 of the shear waves between the sound rays SL1 and SL2 on the basis of the echo signal of the measurement pulse DP which is transmitted to the sound rays SL1 and SL2. The propagation velocity calculating unit 5 calculates propagation velocity V1 of shear wave on the basis of the propagation time difference Δt1 and the distances of the sound rays SL1 and SL2.

The propagation velocity calculating unit 5 calculates a propagation time difference Δt2 of the shear waves between the sound rays SL3 and SL4 on the basis of the echo signal of the measurement pulse DP which is transmitted to the sound rays SL3 and SL4. The propagation velocity calculating unit 5 calculates propagation velocity V2 of shear wave on the basis of the propagation time difference Δt2 and the distances of the sound rays SL3 and SL4. From the above, the propagation velocities V1 and V2 of the shear waves in two places whose distances from the pushing pulse PP are equal to each other.

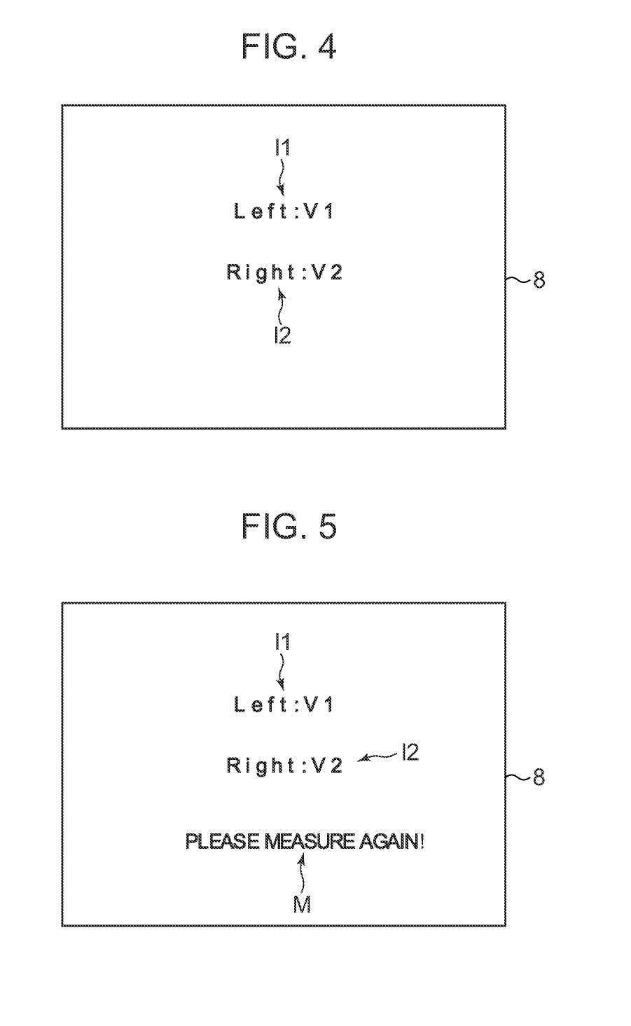

In step S3, as illustrated in FIG. 4, the display control unit 7 makes propagation velocity information I1 and I2 indicative of the propagation velocities V1 and V2 calculated in the step S2 displayed on the display unit 8.

In a place which is not influenced by attenuation of the shear wave and the pushing pulse, ideally, the propagation velocities V1 and V2 are equal to each other. On the other hand, in a region close to a blood vessel or a bone or too far from the body surface, an error between the propagation velocities V1 and V2 becomes large. To compare the propagation velocities V1 and V2, the comparing unit 6 calculates the difference D between the propagation velocities V1 and V2. The display control unit 7 displays the propagation velocity information I1 and I2 in a color according to the difference D. For example, when the difference D is less than n % of the propagation velocity V1 or V2, the display control unit 7 displays the propagation velocity information I1 and I2 in green. When the difference D is equal to or higher than n % and less than m % (n<m) of the propagation velocity V1 or V2, the display control unit 7 displays the propagation velocity information I1 and I2 in yellow. When the difference D is equal to or higher than m % of the propagation velocity V1 or V2, the display control unit 7 displays the propagation velocity information I1 and I2 in red.

Since the difference D between the propagation velocities V1 and V2 calculated is large and accurate propagation velocity is not obtained, “m” is set to a value at which it may be preferable to change the position of the region R. Therefore, in the case where the propagation velocity information I1 and I2 is displayed in red, it means that it is better to change the position of the region R and perform measurement again.

“n” is set to a value that it is necessary to draw attention on accuracy of the propagation velocities V1 and V2 calculated. Therefore, in the case where the propagation velocity information I1 and I2 is displayed in yellow, it means that attention may be required regarding the accuracy of the propagation velocity information I1 and I2.

On the other hand, in the case where the propagation velocity information I1 and I2 is displayed in green, it means that the propagation velocity information I1 and I2 is reliable information.

The colors of the propagation velocity information I1 and I2 are an example of the embodiment of the notification on the basis of a comparison result by the comparing unit. The display control unit 7 is an example of the embodiment of the notifying unit.

In the case where the difference D is equal to or higher than m % of the propagation velocity V1 or V2, the display control unit 7 may display the propagation velocity information I1 and I2 in red and display a message M urging re-measurement as illustrated in FIG. 5. The message M is an example of an example of notification based on a comparison result by the comparing unit.

According to the foregoing embodiment, by the display of the propagation velocity information I1 and I2, the propagation velocity having a correlation with the elasticity of a living tissue can be known. Consequently, information on the elasticity of the living tissue can be known.

From the color of the propagation velocity information I1 and I2 and the message M, whether re-measurement is better to be performed while changing the position of the region R or not can be known. Therefore, the operator can perform measurement in a position where more accurate elasticity can be known, so that more accurate information on the elasticity of a living tissue can be known.

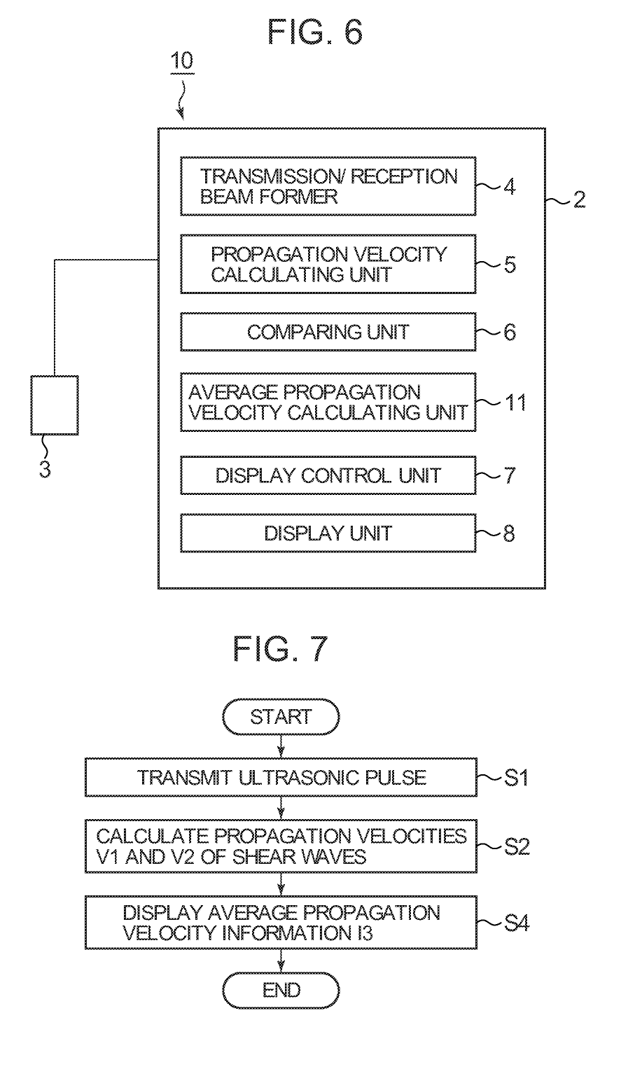

Next, modifications of the first embodiment will be described. A first modification will be described. The device body 2 of the measuring device 10 of the modification has, as illustrated in FIG. 6, the transmission/reception beam former 4, the propagation velocity calculating unit 5, the comparing unit 6, the display control unit 7, and the display unit 8 and, in addition, an average propagation velocity calculating unit 11. The average propagation velocity calculating unit 11 calculates an average value Vav of the propagation velocities V1 and V2. The average propagation velocity calculating unit 11 is an example of an embodiment of an average propagation velocity calculating unit.

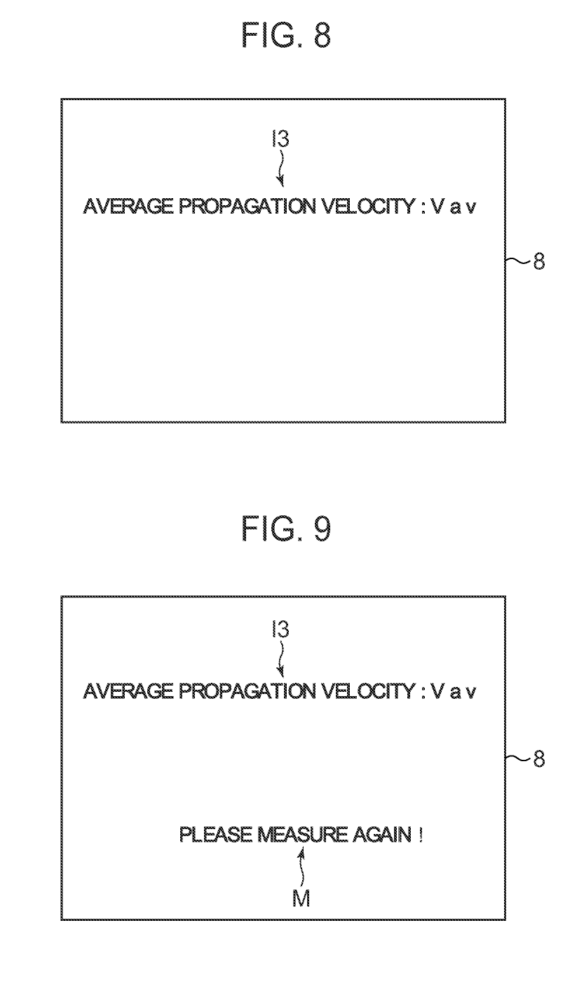

The operation of the modification will be described on the basis of the flowchart of FIG. 7. When the propagation velocities V1 and V2 are calculated in step S2, the routine advances to the process in step S4. In the step S4, as illustrated in FIG. 8, the display control unit 7 displays the average propagation velocity information 13 indicative of the average value Vav.

In a manner similar to the above, the display control unit 7 displays the average propagation velocity information I3 in a color according to the difference D. In the case where the error D is m % or higher, the display control unit 7 may display the message M as illustrated in FIG. 9.

In the above-described modification, by the display of the average propagation velocity information I3, propagation velocity having a correlation with the elasticity of a living tissue can be known. Consequently, information on the elasticity of the living tissue can be known. From the color of the average propagation velocity information I3 and the message M, whether re-measurement is better to be performed while changing the position of the region R or not can be known.

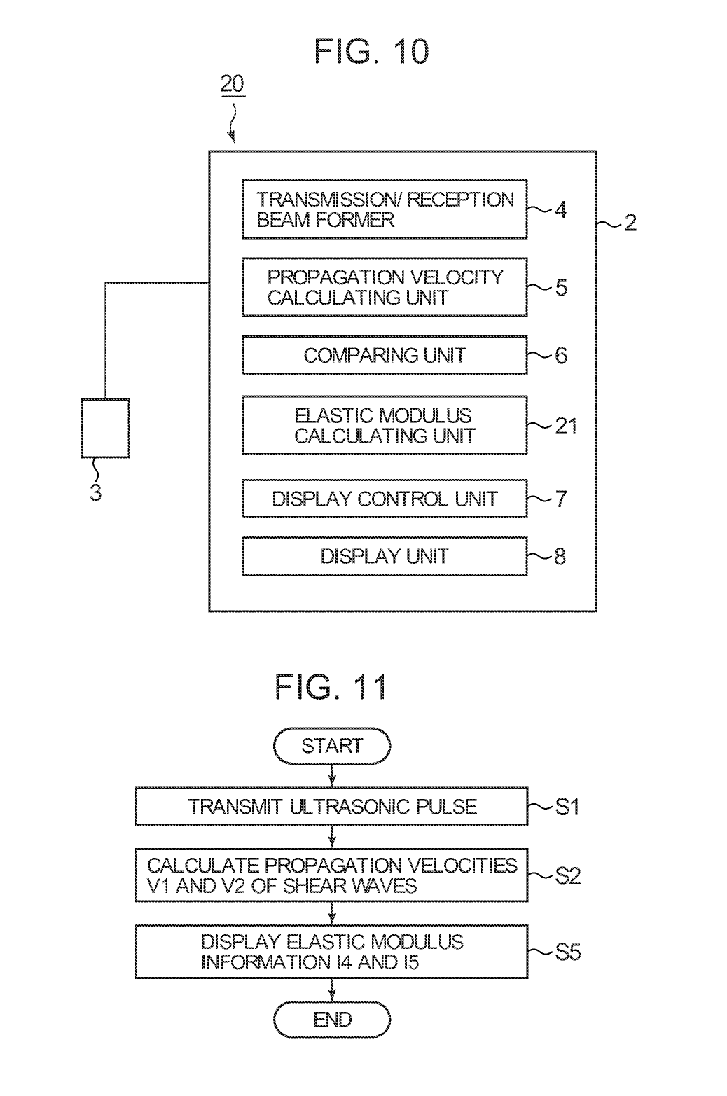

A second modification will now be described. The device body 2 of a measuring device 20 of the modification has, as illustrated in FIG. 10, the transmission/reception beam former 4, the propagation velocity calculating unit 5, the comparing unit 6, the display control unit 7, and the display unit 8 and, in addition, an elastic modulus calculating unit 21. The elastic modulus calculating unit 21 calculates elastic modulus by a known calculation equation on the basis of the propagation velocity V calculated by the propagation velocity calculating unit 5. In the modification, the elastic modulus calculating unit 21 calculates elastic modulus E1 on the basis of the propagation velocity V1. The elastic modulus calculating unit 21 calculates elastic modulus E2 on the basis of the propagation velocity V2.

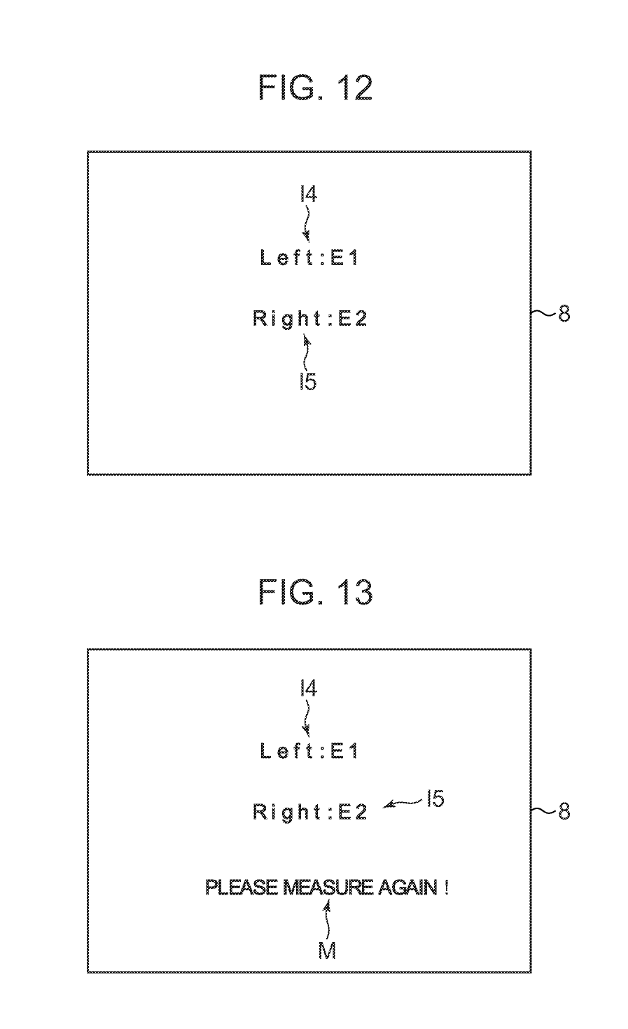

The operation of the modification will be described on the basis of the flowchart illustrated in FIG. 11. When the propagation velocities V1 and V2 of the shear waves are calculated in the step S2, the routine advances to the process in step S5. In step S5, first, the elastic modulus calculating unit 21 calculates the elastic moduli E1 and E2. The display control unit 7 displays, as illustrated in FIG. 12, elasticity modulus information I4 and I5 indicative of the elasticity moduli E1 and E2 on the display unit 8.

The display control unit 7 displays the elastic modulus information I4 and I5 in a color according to the difference D of the propagation velocities V1 and V2 in a manner similar to the above. The display control unit 7 may display the message M as illustrated in FIG. 13.

In the second modification, the comparing unit 6 may calculate the difference D′ of the elastic moduli E1 and E2 to compare the elastic moduli E1 and E2. The display control unit 7 may display the elastic modulus information I4 and I5 in a color according to the difference D′.

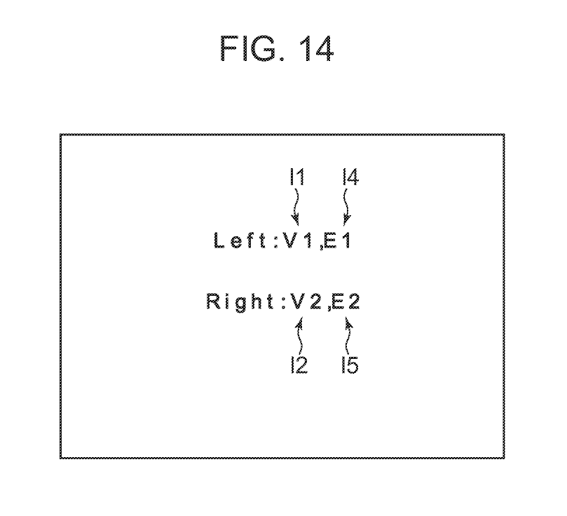

As illustrated in FIG. 14, the display control unit 7 may display the propagation velocity information I1 and I2 together with the elastic modulus information I4 and I5 in a color according to the difference D or D′.

Further, the display control unit 7 may display the information while switching the propagation velocity information I1 and I2 and the elastic modulus information I4 and I5 on the basis of an input of an operation unit (not illustrated) by an operator.

According to the modification described above, by the display of the elastic modulus information I4 and I5, the elasticity of a living tissue can be known. From the color of the elastic modulus information I4 and I5 and the message M, whether re-measurement is necessary while changing the position of the region R or not can be known.

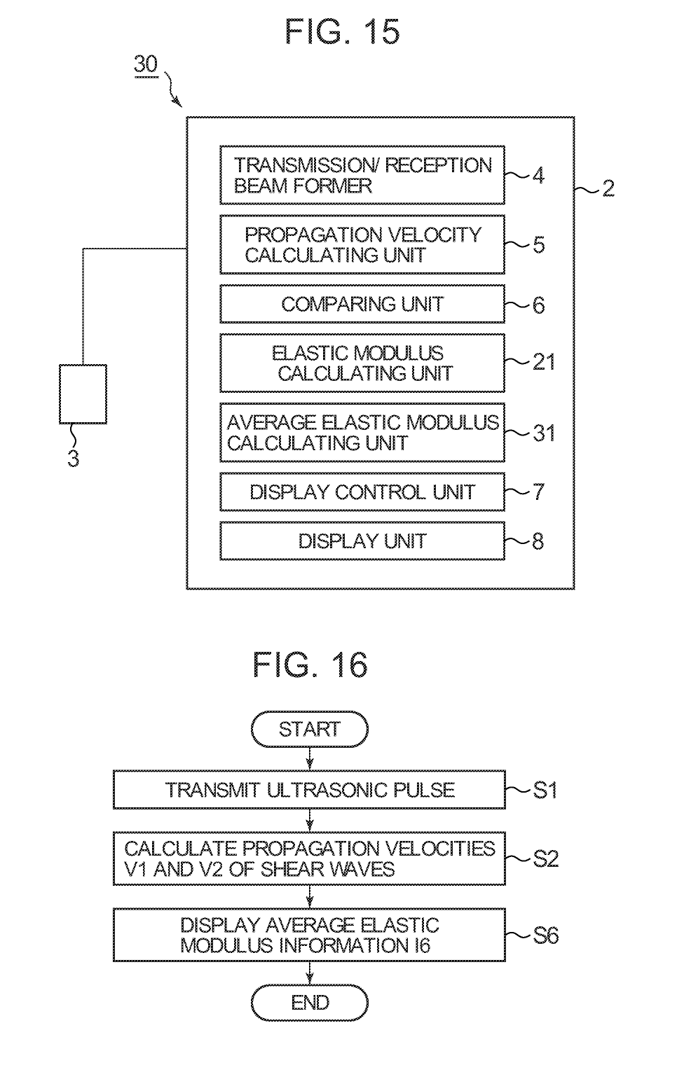

A third modification will be described. The device body 2 of a measuring device 30 of the modification has, as illustrated in FIG. 15, the transmission/reception beam former 4, the propagation velocity calculating unit 5, the comparing unit 6, the elastic modulus calculating unit 21, the display control unit 7, and the display unit 8 and, in addition, an average elastic modulus calculating unit 31. The average elastic modulus calculating unit 31 calculates an average value Eav of elastic moduli in a plurality of parts. The average elastic modulus calculating unit 31 is an example of an embodiment of an average elastic modulus calculating unit.

The average elastic modulus calculating unit 31 may calculate an average value Eav from the elastic moduli E1 and E2. The average elastic modulus calculating unit 31 may calculate an average value Vav of the propagation velocities V1 and V2 calculated by the propagation velocity calculating unit 5 and calculate an elastic modulus from the average value Vav. That is, the elastic modulus calculated from the average value Vav may be used as the average value Eav.

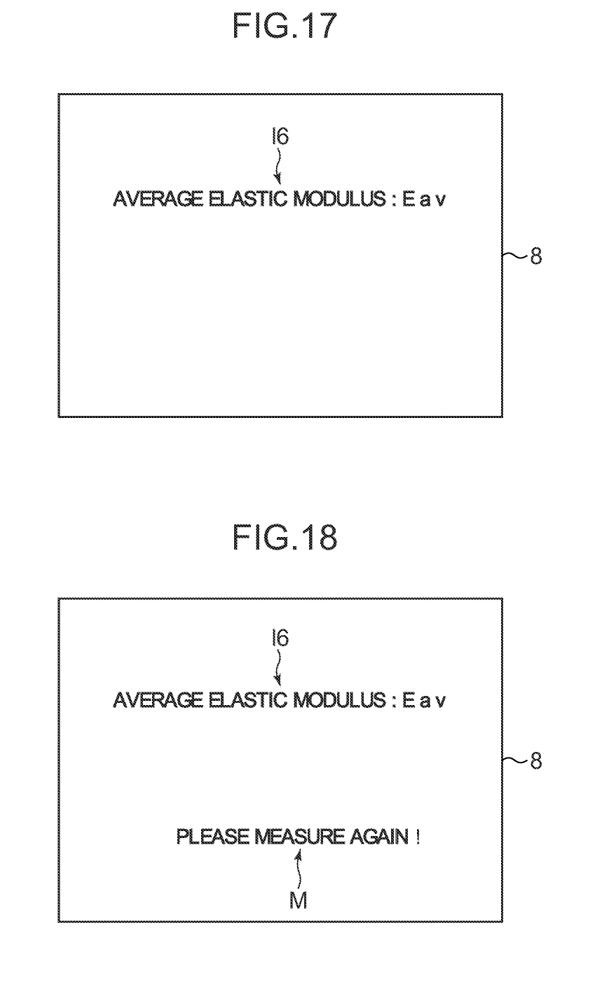

The operation of the modification will be described on the basis of the flowchart of FIG. 16. When the propagation velocities V1 and V2 of the shear waves are calculated in the step S2, the routine advances to the process in step S6. In step S6, first, the elastic modulus calculating unit 21 calculates the elastic moduli E1 and E2. Next, the average elastic modulus calculating unit 31 calculates the average value Eav from the elasticity moduli E1 and E2. The display control unit 7 displays, as illustrated in FIG. 17, average elasticity modulus information I6 indicative of the average value Eav on the display unit 8.

The average elastic modulus calculating unit 31 may calculate the average value Eav from the average value Vav as described above. In this case, the elastic modulus calculating unit 21 may not always have to calculate the elastic moduli E1 and E2.

The display control unit 7 displays the elastic modulus information I6 in a color according to the difference D of the propagation velocities V1 and V2. The display control unit 7 may display the message M as illustrated in FIG. 18.

In a manner similar to the second modification, the display control unit 7 may display the average elastic modulus information I6 in a color according to the difference D′ of the elastic moduli E1 and E2.

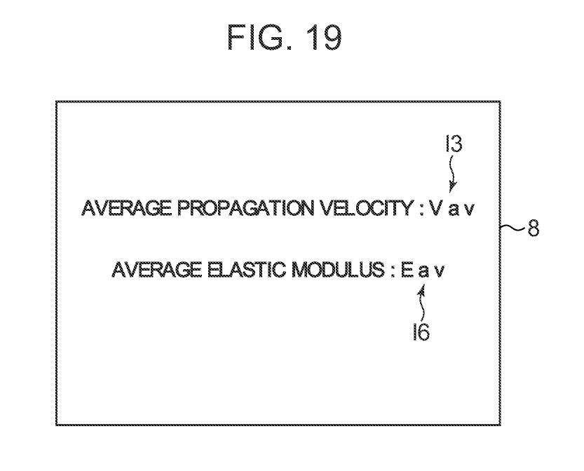

As illustrated in FIG. 19, the display control unit 7 may display the average propagation velocity information I3 together with the average elastic modulus information I6 in a color according to the difference D or D′. In this case, although not illustrated, it is assumed that the device body 2 has the average propagation velocity calculating unit 11.

The display control unit 7 may display the information while switching the average propagation velocity information I3 and the average elastic modulus information I6 on the basis of an input of an operation unit (not illustrated) by an operator.

According to the modification described above, by the display of the average elastic modulus information I6, the elasticity of a living tissue can be known. From the color of the average elastic modulus information I6 and the message M, whether re-measurement is necessary while changing the position of the region R or not can be known.

### Second Embodiment

A second embodiment will now be described. Description of the same items as those of the first embodiment will not be repeated.

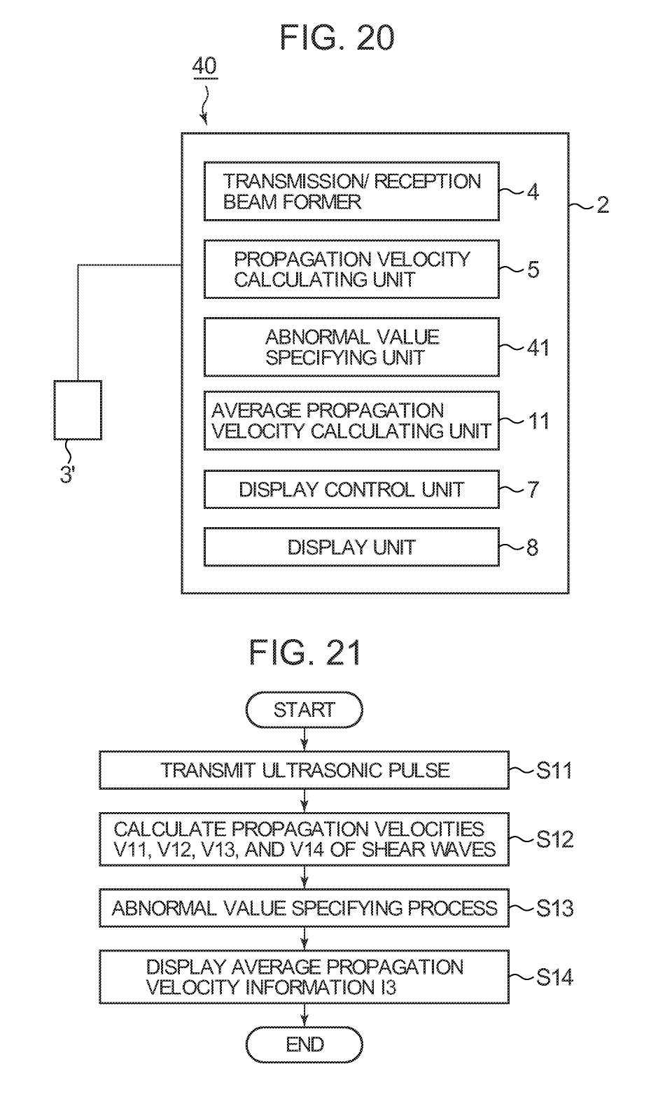

The device body 2 of a measuring device 40 of the second embodiment has, as illustrated in FIG. 20, the transmission/reception beam former 4, the propagation velocity calculating unit 5, an abnormal value specifying unit 41, the average propagation velocity calculating unit 11, the display control unit 7, and the display unit 8. The abnormal value specifying unit 41 is an example of an embodiment of an abnormal value specifying unit.

In the embodiment, an ultrasonic probe 3′ is a 2D array probe in which ultrasonic transducers arranged in the azimuth direction (X direction) and an elevation direction (Z direction) which are orthogonal to each other.

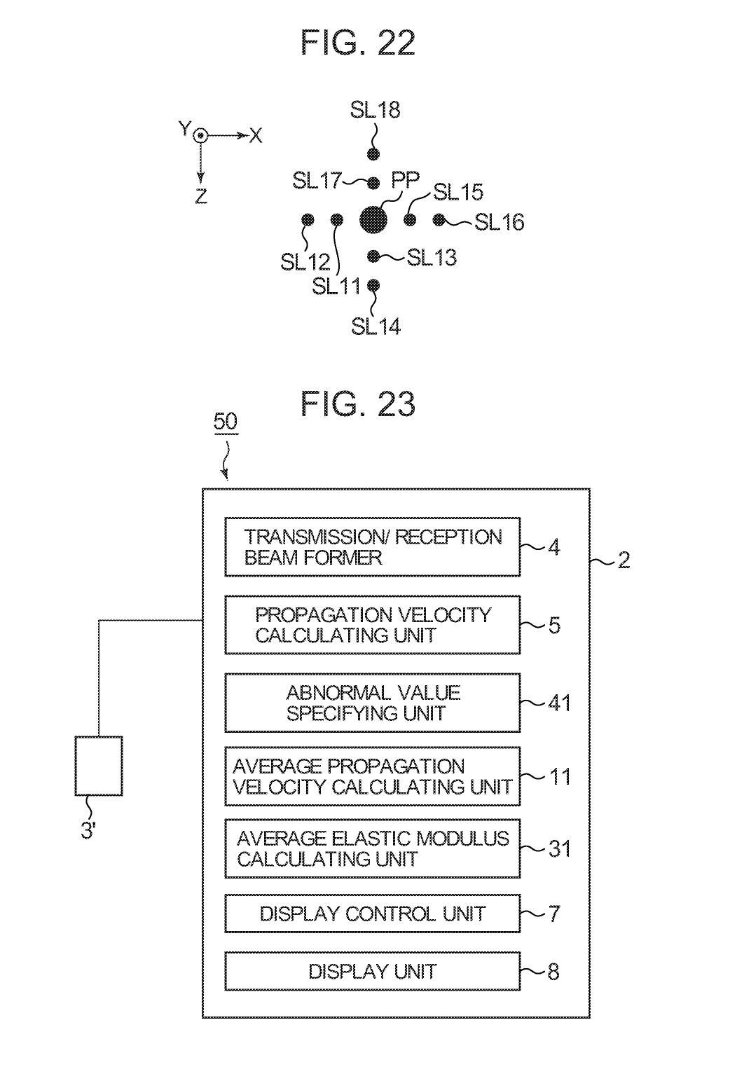

The operation of the modification will be described on the basis of the flowchart of FIG. 21. In transmission of the ultrasonic pulse in step S11, basically, like in the step S1, ultrasonic pulses are transmitted. However, the number of the measurement pulses is different. In step S11, as illustrated in FIG. 22, the measurement pulse DP is transmitted on sound rays SL11 and SL12, sound rays SL13 and SL14, sound rays SL15 and SL16, and sound rays SL17 and SL18 around the pushing pulse PP. FIG. 22 is a plan view seen from the transmission/reception direction (Y-axis direction) of ultrasonic waves.

The distances from the pushing pulse PP to the sound rays SL11, SL13, SL15, and S17 are equal to one another. The distances from the pushing pulse PP to the sound rays SL12, SL14, SL16, and SL18 are equal to one another. The distance between the sound rays SL11 and SL12, the distance between the sound rays SL13 and SL14, the distance between the sound rays SL15 and SL16, and the distance between the SL17 and SL18 are equal to one another.

Next, in step S12, the propagation velocity calculating unit 5 calculates propagation velocity V of the shear wave. The propagation velocity calculating unit 5 calculates a propagation time difference Δt1 of shear waves between the sound rays SL11 and SL12 on the basis of the echo signal of the measurement pulse DP transmitted to the sound rays SL11 and SL12. The propagation velocity calculating unit 5 calculates propagation velocity V11 of the shear wave on the basis of the propagation time difference Δt1 and the distance between the sound rays SL11 and SL12.

The propagation velocity calculating unit 5 calculates propagation velocity V12 of the shear wave in a manner similar to the above on the basis of echo signals of the measurement pulse DP transmitted to the sound rays SL13 and SL14, and calculates propagation velocity V13 of the shear wave in a manner similar to the above on the basis of echo signals of the measurement pulse DP transmitted to the sound rays SL15 and SL16. Further, the propagation velocity calculating unit 5 calculates propagation velocity V14 of the shear wave in a manner similar to the above on the basis of echo signals of the measurement pulse DP transmitted to the sound rays SL17 and SL18. From the above, the propagation velocities V11, V12, V13, and V14 of the shear waves in four places whose distances from the pushing pulse PP are equal are calculated.

In step S13, the abnormal value specifying unit 41 performs abnormal value specifying process. Specifically, it is determined whether the propagation velocities V11, V12, V13, and V14 calculated in the step S12 are values considerably different from the other values or not. For example, the abnormal value specifying unit 41 determines a value which seems to exceed the range of a measurement error as an abnormal value. It is assumed that the criterion of whether a value is considerably different or not is preset.

In step S14, like in FIG. 8, the average propagation velocity information I3 is displayed in the display unit 8. Concretely, first, the average propagation velocity calculating unit 11 calculates the average value Vav of the propagation velocities V other than the abnormal value specified in the step S13. When it is assumed that the propagation velocity V13 is an abnormal value, the average propagation velocity calculating unit 11 calculates the average value Vav of the propagation velocities V11, V12, and V14. The display control unit 8 displays the average propagation velocity information I3 indicative of the average value Vav on the display unit 8.

In the above-described embodiment, the average propagation velocity information I3 indicative of the average value Vav calculated while the abnormal value is excluded is displayed, so that more accurate information on the elasticity of a living tissue can be known.

Next, modifications of the second embodiment will be described. A first modification will be described. The device body 2 of a measuring device 50 of the modification has, as illustrated in FIG. 23, the transmission/reception beam former 4, the propagation velocity calculating unit 5, the abnormal value specifying unit 41, the average propagation velocity calculating unit 11, the display control unit 7, and the display unit 8 and, in addition, the average elastic modulus calculating unit 31.

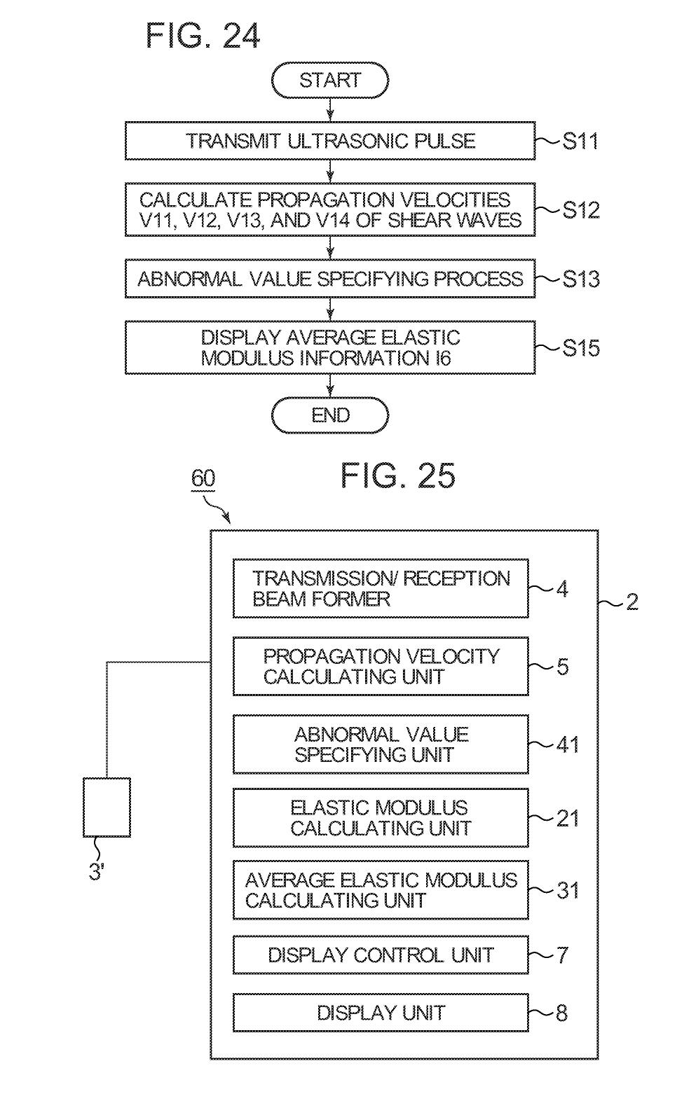

The operation of the modification will be described on the basis of the flowchart of FIG. 24. After the abnormal value specifying process is performed in the step S13, the routine advances to the process in step S15, and average elastic modulus information I6 is displayed. Specifically, in step S15, first, like in the step S14, the average propagation velocity calculating unit 11 calculates the average value Vav of the propagation velocities other than the abnormal value specified in the step S13.

Next, the average elastic modulus calculating unit 31 calculates an elastic modulus from the average value Vav. The display control unit 7 displays the average elastic modulus information I6 indicative of the elastic modulus in the display unit 8 like in FIG. 17. The display control unit 7 may display the average propagation velocity information I3 together with the average elastic modulus information I6 like in FIG. 19.

According to the modification described above, by the display of the average elastic modulus information I6 indicative of the elastic modulus calculated from the average value Vav of the propagation velocities calculated while an abnormal value is excluded, more accurate elastic modulus can be known.

Next, a second modification will be described. A measuring device 60 of the modification has, as illustrated in FIG. 25, the transmission/reception beam former 4, the propagation velocity calculating unit 5, the abnormal value specifying unit 41, the average elastic modulus calculating unit 31, the display control unit 7, and the display unit 8 and, in addition, the elastic modulus calculating unit 21.

The operation of the modification will be described. Also in the modification, processes similar to those of the flowchart of FIG. 24 are basically performed. However, a concrete process in step S15 is different. In the modification, the elastic modulus calculating unit 21 calculates an elastic modulus on the basis of each of the propagation velocities V other than the abnormal value specified in the step S13. When it is assumed that the propagation velocity V13 in the propagation velocities V11, V12, V13, and V14 is an abnormal value, the elastic modulus calculating unit 21 calculates an elastic modulus E11 on the basis of the propagation velocity V11, calculates an elastic modulus E12 on the basis of the propagation velocity V12, and calculates an elastic modulus E14 on the basis of the propagation velocity V14.

The average elastic modulus calculating unit 31 calculates an average value Eav of the elastic moduli E11, E12, and E14. The display control unit 7 displays average elastic modulus information I6 indicative of the average value Eav in the display unit 8 like in FIG. 17.

According to the modification described above, the elastic moduli E11, E12, and E14 are calculated from the propagation velocities except for the abnormal value, and the average elastic modulus information I6 indicative of the average value Eav is displayed, so that more accurate elastic modulus can be known.

A third modification will now be described. A measuring device of the modification has the configuration of FIG. 25 in a manner similar to the second modification.

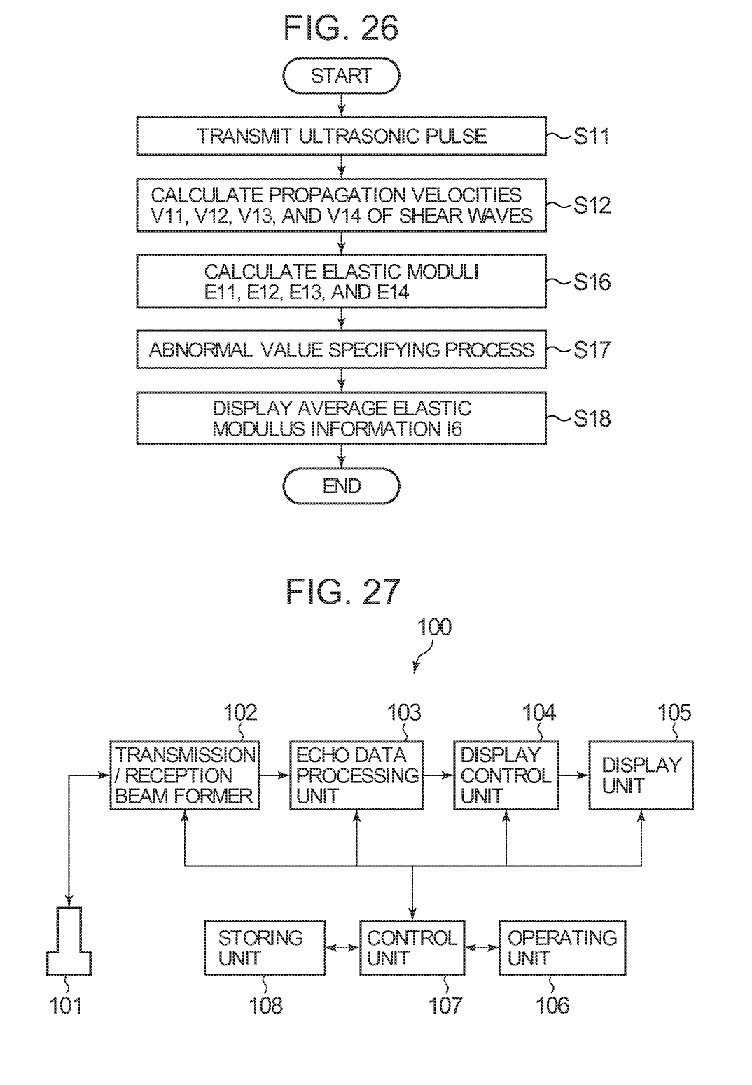

The operation of the modification will be described on the basis of the flowchart of FIG. 26. In the modification, when the propagation velocities V11, V12, V13, and V14 are calculated in the step S12, the routine advances to the process in step S16. In the process of the step S16, the elastic modulus calculating unit 21 calculates the elastic moduli E11, E12, E13, and E14 on the basis of the propagation velocities V11, V12, V13, and V14.

In step S17, the abnormal value specifying unit 41 performs an abnormal value specifying process. In the modification, the abnormal value specifying unit 41 determines whether each of the elastic moduli E11, E12, E13, and E14 calculated in the step S16 is considerably different from the other values or not. For example, the abnormal value specifying unit 41 determines a value which seems to exceed the range of a measurement error as an abnormal value. It is assumed that the criterion of whether a value is considerably different or not is preset.

In step S18, like in FIG. 8, the average elastic modulus information I6 is displayed in the display unit 8. Specifically, first, the average elastic modulus calculating unit 31 calculates the average value Eav of the elastic modulus E other than the abnormal value specified in the step S17. When it is assumed that the elastic modulus E13 is an abnormal value, the average elastic modulus calculating unit 31 calculates the average value Eav of the elastic moduli E11, E12, and E14. The display control unit 7 displays the average elastic modulus information I6 indicative of the average value Eav on the display unit 8.

In the above-described embodiment, the average elastic modulus information I6 indicative of the average value Eav calculated from the elastic moduli E11, E12, and E 14 except for the abnormal value is displayed, so that more accurate elastic modulus can be known.

### Third Embodiment

Next, a third embodiment will be described. In the embodiment, an ultrasonic diagnostic apparatus including the measuring device of the first embodiment will be described.

As illustrated in FIG. 27, an ultrasonic diagnostic apparatus 100 has an ultrasonic probe 101, a transmission/reception beam former 102, an echo data processing unit 103, a display control unit 104, a display unit 105, an operating unit 106, a control unit 107, and a storing unit 108.

The ultrasonic probe 101 and the transmission/reception beam former 102 have a configuration similar to that of the ultrasonic probe 3 and the transmission/reception beam former 4, so that their description will not be repeated.

The echo data processing unit 103 performs a signal process for generating an ultrasonic image on echo data which is output from the transmission/reception beam former 102. For example, the echo data processing unit performs a B-mode process such as a logarithmic compression process, or an envelope detecting process to generate B-mode data.

The display control unit 104 displays an ultrasonic image on the display unit 105 on the basis of data entered from the echo data processing unit 103. For example, the display control unit 104 performs a scan conversion by a scan converter on the B-mode data to generate B-mode image data and displays a B-mode image on the basis of the B-mode image data.

Like the display control unit 7, the display control unit 104 displays propagation velocity information indicative of the propagation velocity V on the display unit 8. The display control unit 104 is also an example of an embodiment of a notifying unit.

Like the display unit 8, the display unit 105 is, for example, an LCD (Liquid Crystal Display), a CRT (Cathode Ray Tube), or the like. The operating unit 106 is constructed by including a keyboard and a pointing device (not illustrated) used by the operator to enter an instruction and information.

The control unit 107 is constructed by a CPU (Central Processing Unit) and executes the functions in the units in the ultrasonic diagnostic apparatus 100.

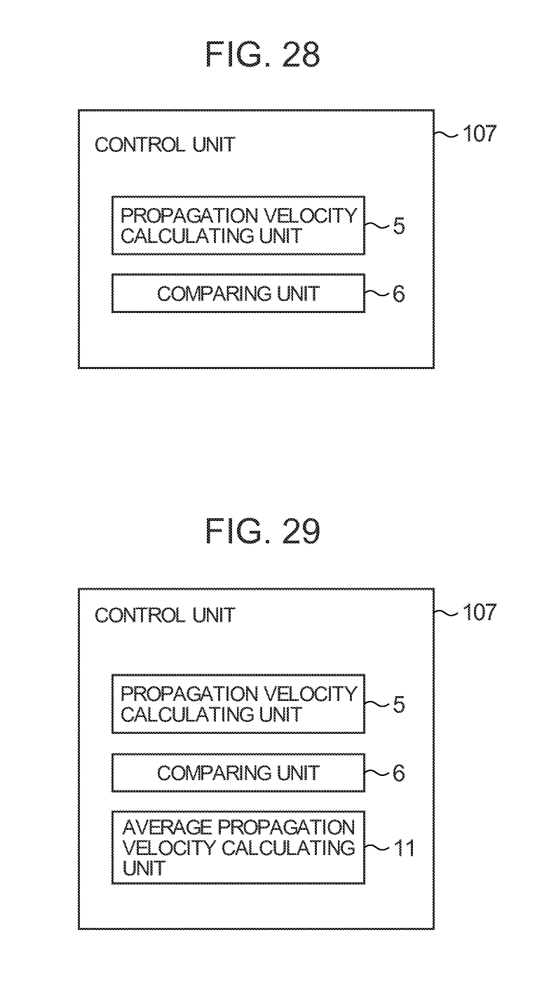

The control unit 107 has, as illustrated in FIG. 28, the propagation velocity calculating unit 5 and the comparing unit 6 described in the first embodiment.

The storage unit 10 is, for example, a semiconductor memory such as an HDD (Hard Disk Drive), a RAM (Random Access Memory), or a ROM (Read Only Memory).

The operation of the ultrasonic diagnostic apparatus 1 of the embodiment will be described. Also in the ultrasonic diagnostic apparatus 1 of the embodiment, processes similar to those of the flowchart of FIG. 2 are performed. Specifically, in step S1, the pushing pulse PP and the measurement pulse DP are transmitted from the ultrasonic probe 102. In step S2, the propagation velocity calculating unit 5 calculates the propagation velocities V1 and V2 of shear waves.

In step S3, the display control unit 104 displays the propagation velocity information I1 and I2 in a color according to the difference D calculated by the comparing unit 6 on the display unit 8 (refer to FIG. 4). Although not illustrated, an ultrasonic image such as a B-mode image may be displayed together with the propagation velocity information I1 and I2.

The ultrasonic diagnostic apparatus 100 of the embodiment described above can obtain effects similar to those of the first embodiment.

Modifications of the third embodiment will be described. A first modification will be described. The ultrasonic diagnostic apparatus 100 of the first modification corresponds to the first modification of the first embodiment. The control unit 107 has, as illustrated in FIG. 29, the propagation velocity calculating unit 5 and the comparing unit 6 and, in addition, the average propagation velocity calculating unit 11.

In the modification, the processes in the flowchart of FIG. 7 are performed. In the step S4, the display control unit 104 displays the average propagation velocity information I3 in a color according to the difference D (refer to FIG. 8). At this time, an ultrasonic image may be displayed together with the average propagation velocity information I3. The display control unit 104 may display the message M (refer to FIG. 9).

Also by the modification, effects similar to those of the first modification of the first embodiment can be obtained.

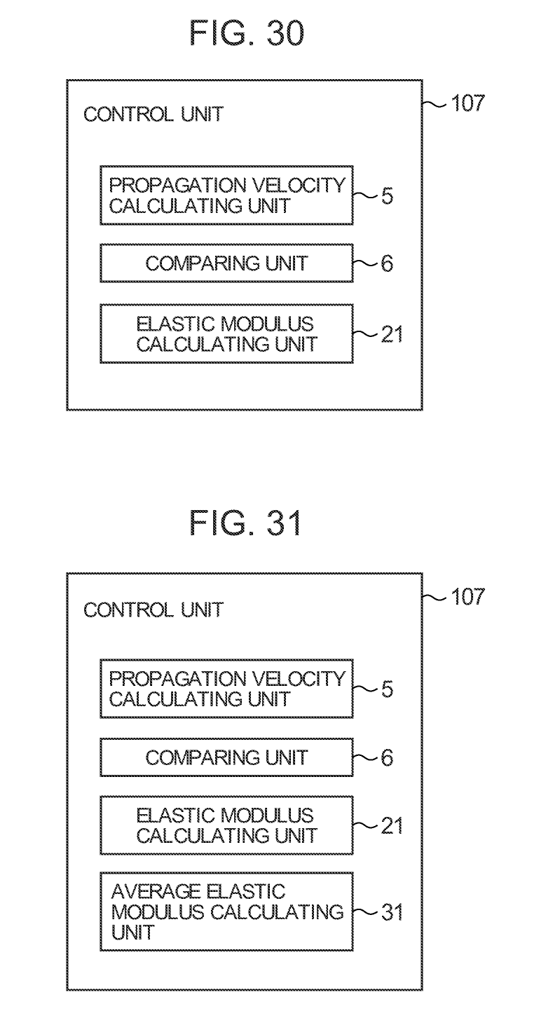

Next, a second modification will be described. The ultrasonic diagnostic apparatus 100 of the second modification corresponds to the second modification of the first embodiment. The control unit 107 has, as illustrated in FIG. 30, the propagation velocity calculating unit 5 and the comparing unit 6 and, in addition, the elastic modulus calculating unit 21.

In the modification, the processes in the flowchart of FIG. 11 are performed. In the step S5, the display control unit 104 displays the elastic modulus information I4 and I5 in a color according to the difference D or D′ (refer to FIG. 12). At this time, an ultrasonic image may be displayed together with the elastic modulus information I4 and I5. The display control unit 104 may display the message M (refer to FIG. 13). The propagation velocity information I1 and I2 may be displayed together with the elastic modulus information I4 and I5 (refer to FIG. 14).

Also by the modification, effects similar to those of the second modification of the first embodiment can be obtained.

Next, a third modification will now be described. The ultrasonic diagnostic apparatus 100 of the third modification corresponds to the third modification of the first embodiment. The control unit 107 has, as illustrated in FIG. 31, the propagation velocity calculating unit 5, the comparing unit 6, and the elastic modulus calculating unit 21 and, in addition, the average elastic modulus calculating unit 31.

In the modification, the processes in the flowchart of FIG. 16 are performed. In the step S6, the display control unit 104 displays the average elastic modulus information I6 in a color according to the difference D or D′ (refer to FIG. 17). At this time, an ultrasonic image may be displayed together with the average elastic modulus information I6. The display control unit 104 may display the message M (refer to FIG. 18). The average propagation velocity information I3 may be displayed together with the average elastic modulus information I6 (refer to FIG. 19). In this case, although not illustrated, it is assumed that the control unit 107 has the average propagation velocity calculating unit 11.

The display control unit 104 may display the average propagation velocity information I3 together with the average elastic modulus information I6 or may display the information I3 and I6 while switching the information I3 and I6.

Also by the modification, effects similar to those of the third modification of the first embodiment can be obtained.

### Fourth Embodiment

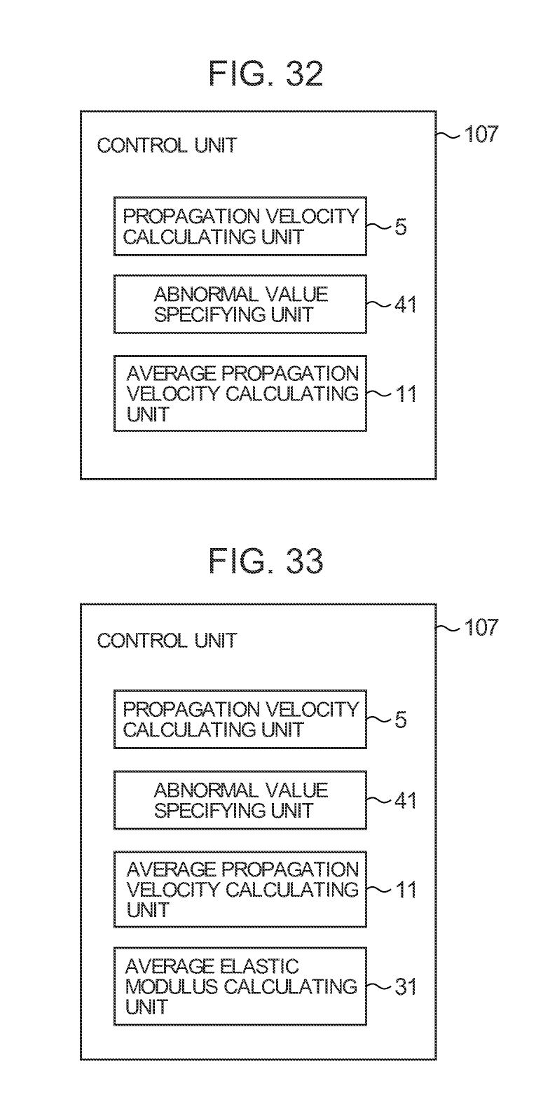

Next, a fourth embodiment will be described. An ultrasonic diagnostic apparatus of the fourth embodiment has the same basic configuration as that of the ultrasonic diagnostic apparatus 100 of the third embodiment illustrated in FIG. 27. The ultrasonic diagnostic apparatus of the embodiment includes the measuring device of the second embodiment. Therefore, the ultrasonic probe 101 (refer to FIG. 27) is a 2D array probe like the ultrasonic probe 3′. The control unit 107 has, as illustrated in FIG. 32, the propagation velocity calculating unit 5, the abnormal value specifying unit 41, and the average propagation velocity calculating unit 11.

In the embodiment, the processes in the flowchart of FIG. 21 are performed. In the step S14, the display control unit 104 displays the average propagation velocity information I3 indicative of the average value Vav calculated while excluding an abnormal value (refer to FIG. 8). At this time, an ultrasonic image may be displayed together with the average propagation velocity information I3. The display control unit 104 may display the message M (refer to FIG. 9).

Also by the embodiment, effects similar to those of the second embodiment can be obtained.

Next, modifications of the fourth embodiment will be described. A first modification will be described. An ultrasonic diagnostic apparatus of the first modification corresponds to the first modification of the second embodiment. The control unit 107 has, as illustrated in FIG. 33, the propagation velocity calculating unit 5, the abnormal value specifying unit 41, and the average propagation velocity calculating unit 11 and, in addition, the average elastic modulus calculating unit 31.

In the modification, the processes in the flowchart of FIG. 24 are performed. In the step S15, the display control unit 104 displays the average elastic modulus information I6 (refer to FIG. 17). At this time, an ultrasonic image may be displayed together with the average elastic modulus information I6. The average propagation velocity information I3 may be displayed together with the average elastic modulus information I6 (refer to FIG. 19).

Also by the modification, effects similar to those of the first modification of the second embodiment can be obtained.

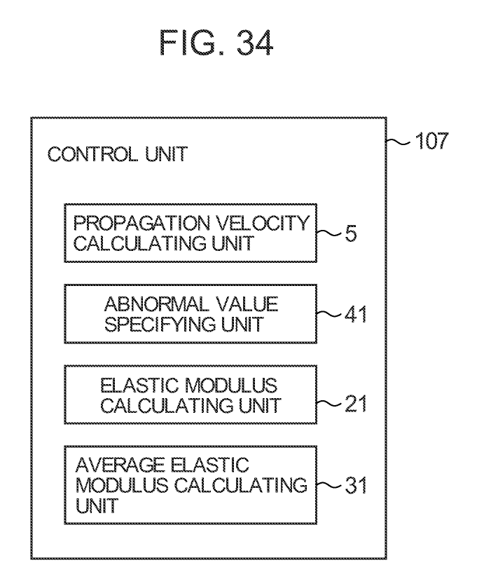

Next, a second modification will be described. An ultrasonic diagnostic apparatus of the second modification corresponds to the second modification of the second embodiment. The control unit 107 has, as illustrated in FIG. 34, the propagation velocity calculating unit 5, the abnormal value specifying unit 41, and the average elastic modulus calculating unit 31 and, in addition, the elastic modulus calculating unit 21.

Also in the modification, the processes in the flowchart of FIG. 24 are performed. In the step S15, the display control unit 104 displays the average elastic modulus information I6 (refer to FIG. 17). At this time, an ultrasonic image may be displayed together with the average elastic modulus information I6.

Also by the modification, effects similar to those of the second modification of the second embodiment can be obtained.

Next, a third modification will now be described. An ultrasonic diagnostic apparatus of the third modification corresponds to the third modification of the second embodiment. In a manner similar to the second embodiment, the control unit 107 has the configuration of FIG. 34.

In the modification, the processes in the flowchart of FIG. 26 are performed. In the step S18, the display control unit 104 displays the average elastic modulus information I6 (refer to FIG. 17). At this time, an ultrasonic image may be displayed together with the average elastic modulus information I6.

Also by the modification, effects similar to those of the third modification of the second embodiment can be obtained.

Although the disclosure has been described by the foregoing exemplary embodiments, obviously, the systems and methods described herein can be variously modified without changing the gist of the invention. For example, the comparing unit 6 may calculate the ratio between the propagation velocities V1 and V2 in place of the difference D between the propagation velocities V1 and V2. Also in the first embodiment, like in the second embodiment, the ultrasonic probe 3 may be a 2D array probe. In this case, in a manner similar to the second embodiment, the measurement pulse DP may be transmitted onto eight sound rays around the pushing pulse PP. The number of sound rays maybe larger than eight.

In the first and third embodiments, the information I1 to I6 is displayed in a color according to the difference D or D′. When the difference D or D′ exceeds a predetermined threshold, it can be notified by sound.

1. A measuring device comprising:
a propagation velocity calculating unit configured to calculate a propagation velocity of a shear wave generated in a living tissue by an ultrasonic pulse transmitted to the living tissue, wherein the propagation velocity calculating unit is configured to calculate propagation velocities of shear waves in a plurality of parts whose distances from the ultrasonic pulse are equal;
a comparing unit configured to compare the propagation velocities of the shear waves in the plurality of parts; and
a notifying unit configured to perform a notification based on a result of the comparison performed by the comparing unit.

2. A measuring device comprising:
a propagation velocity calculating unit configured to calculate a propagation velocity of a shear wave generated in a living tissue by an ultrasonic pulse transmitted to the living tissue, wherein the propagation velocity calculating unit is configured to calculate propagation velocities of shear waves in a plurality of parts whose distances from the ultrasonic pulse are equal;
an elastic modulus calculating unit configured to calculate an elastic modulus of the living tissue corresponding to each of the propagation velocities based on the propagation velocities of the shear waves;
a comparing unit configured to compare the plurality of elastic moduli; and
a notifying unit configured to perform a notification based on a result of the comparison performed by the comparing unit.

3. The measuring device according to claim 1, further comprising a display unit configured to display the plurality of propagation velocities calculated by the propagation velocity calculating unit.

4. The measuring device according to claim 2, further comprising a display unit configured to display the plurality of propagation velocities calculated by the propagation velocity calculating unit.

5. The measuring device according to claim 2, further comprising a display unit configured to display the plurality of elastic moduli calculated by the elastic modulus calculating unit.

6. The measuring device according to claim 1, further comprising:
an average propagation velocity calculating unit configured to calculate an average value of propagation velocities of the shear waves in a plurality of parts; and
a display unit configured to display the average value calculated by the average propagation velocity calculating unit.

7. The measuring device according to claim 2, further comprising:
an average propagation velocity calculating unit configured to calculate an average value of propagation velocities of the shear waves in a plurality of parts; and
a display unit configured to display the average value calculated by the average propagation velocity calculating unit.

8. The measuring device according to claim 1, further comprising:
an elastic modulus calculating unit configured to calculate, based on the propagation velocities of the shear waves, an elastic modulus of the living tissue corresponding to each of the propagation velocities; and
a display unit configured to display the plurality of elastic moduli calculated by the elastic modulus calculating unit.

9. The measuring device according to claim 2, further comprising:
an elastic modulus calculating unit configured to calculate, based on the propagation velocities of the shear waves, an elastic modulus of the living tissue corresponding to each of the propagation velocities; and
a display unit configured to display the plurality of elastic moduli calculated by the elastic modulus calculating unit.

10. The measuring device according to claim 1, further comprising:
an average elastic modulus calculating unit configured to calculate an average value of elastic moduli of the living tissue in a plurality of parts based on the propagation velocities of the shear waves; and
a display unit configured to display the average value calculated by the average elastic modulus calculating unit.

11. The measuring device according to claim 2, further comprising:
an average elastic modulus calculating unit configured to calculate an average value of elastic moduli of the living tissue in a plurality of parts; and
a display unit configured to display the average value calculated by the average elastic modulus calculating unit.

12. The measuring device according to claim 10, further comprising:
an average propagation velocity calculating unit configured to calculate an average value of propagation velocities of the shear waves in a plurality of parts; and
a display unit configured to display the average value calculated by the average propagation velocity calculating unit.

13. The measuring device according to claim 11, further comprising:
an average propagation velocity calculating unit configured to calculate an average value of propagation velocities of the shear waves in a plurality of parts; and
a display unit configured to display the average value calculated by the average propagation velocity calculating unit.

14. A measuring device comprising:
a propagation velocity calculating unit configured to calculate propagation velocity of a shear wave generated in a living tissue by an ultrasonic pulse transmitted to the living tissue, wherein the propagation velocity calculating unit is configured to calculate propagation velocities of shear waves in a plurality of parts whose distances from the ultrasonic pulse are equal;
an abnormal value specifying unit configured to specify an abnormal value in accordance with a predetermined reference from the propagation velocities of the plurality of shear waves calculated by the propagation velocity calculating unit; and
an average propagation velocity calculating unit configured to calculate an average value of propagation velocities of the shear waves except for the abnormal value.

15. The measuring device according to claim 10, further comprising a display unit configured to display an average value calculated by the average propagation velocity calculating unit.

16. The measuring device according to claim 14, further comprising an average elastic modulus calculating unit configured to calculate an average elastic modulus on the basis of an average value calculated by the average propagation velocity calculating unit.

17. A measuring device comprising:
a propagation velocity calculating unit configured to calculate propagation velocity of a shear wave generated in a living tissue by an ultrasonic pulse transmitted to the living tissue, wherein the propagation velocity calculating unit is configured to calculate propagation velocities of the shear waves in a plurality of parts whose distances from the ultrasonic pulse are equal;
an abnormal value specifying unit configured to specify an abnormal value in accordance with a predetermined reference from the propagation velocities of the plurality of shear waves calculated by the propagation velocity calculating unit;
an elastic modulus calculating unit configured to calculate elastic moduli of the living tissue in the plurality of parts on the basis of the propagation velocities of the shear waves except for the abnormal value; and
an average elastic modulus calculating unit configured to calculate an average value of the elastic moduli in the plurality of parts.

18. A measuring device comprising:
a propagation velocity calculating unit configured to calculate propagation velocity of a shear wave generated in a living tissue by an ultrasonic pulse transmitted to the living tissue, wherein the propagation velocity calculating unit is configured to calculate propagation velocities of the shear waves in a plurality of parts whose distances from the ultrasonic pulse are equal;
an elastic modulus calculating unit configured to calculate elastic moduli of the living tissue in the plurality of parts on the basis of the propagation velocities of the shear waves;
an abnormal value specifying unit configured to specify an abnormal value in accordance with a predetermined reference from the plurality of elastic moduli; and
an average elastic modulus calculating unit configured to calculate an average value of the plurality of elastic moduli except for the abnormal value.

19. The measuring device according to claim 16, further comprising a display unit configured to display an average elastic modulus calculated by the average elastic modulus calculating unit.

20. The measuring device according to claim 1, wherein the propagation velocity calculating unit is configured to calculate the propagation velocity on the basis of an echo signal of an ultrasonic pulse transmitted to the living tissue separately from the ultrasonic pulse.
